(19) United States
(12) Patent Application Publication
(10) Pub. No.: US 2006/0167045 A1
(43) Pub. Date: Jul. 27, 2006

(54) Combination therapy using a dual ppar alpha/gamma agonist and an angiotensin II type I receptor antagonist

(76) Inventors: Joanne Waldstreicher, Scotch Plains, NJ (US); Jonathan C. Fox, Narberth, PA (US); Michele Mercuri, Scotch Plains, NJ (US)

Correspondence Address: MERCK AND CO., INC, P O BOX 2000, RAHWAY, NJ, 07065-0907, US

(21) Appl. No.: 10/524,865

(22) PCT Filed: Aug. 15, 2003
(86) PCT No.: PCT/US03/25434

§ 371 Date: Feb. 14, 2005

Related U.S. Application Data
(60) Provisional application No. 60/404,963, filed on Aug. 21, 2002.

(51) Int. Cl. A61K 31/4709; A61K 31/427; A61K 31/422; A61K 31/4184; A61K 31/41; A61K 31/192

(52) U.S. Cl. 514/311; 514/369; 514/376; 514/381; 514/569; 514/570; 514/567; 514/561

(57) ABSTRACT

The present invention relates to the treatment of hypertension and type 2 diabetes, Metabolic Syndrome or a pre-diabetic state, by the administration of a therapeutically effective amount of a combination of a dual PPARα/γ agonist and an Angiotensin II type I receptor antagonist, including pharmaceutically acceptable salts and solvates of said active ingredients.

### FIELD OF THE INVENTION

The instant invention is concerned with the use of combinations of pharmaceutically active compounds that are dual agonists of the alpha and gamma subtypes of the peroxisome proliferator activated receptor (PPARα/γ) with other compounds that are active in decreasing hypertension, such as Angiotensin II Type I receptor (A-2) antagonists.

### BACKGROUND OF THE INVENTION

Diabetes refers to a disease process derived from multiple causative factors and is characterized by elevated levels of plasma glucose (hyperglycemia) in the fasting state or following glucose administration during an oral glucose tolerance test. Frank diabetes mellitus (e.g., a blood glucose level ≧126 mg/dL in a fasting state) is associated with increased and premature cardiovascular morbidity and mortality, and is related both directly and indirectly to various metabolic conditions, including alterations of lipid, lipoprotein and apolipoprotein metabolism. Patients with non-insulin dependent diabetes mellitus (type 2 diabetes mellitus) or type-2 diabetes (approximately 95% of patients with diabetes mellitus) frequently display elevated levels of serum lipids, such as cholesterol and triglycerides, and have poor blood-lipid profiles, with high levels of LDL-cholesterol and low levels of HDL-cholesterol. Those suffering from Type 2 diabetes mellitus are thus at an especially increased risk of developing macrovascular and microvascular complications, including coronary heart disease, stroke, peripheral vascular disease, hypertension (for example, blood pressure ≧130/80 mmHg in a resting state), nephropathy, neuropathy and retinopathy.

Patients having type 2 diabetes mellitus characteristically exhibit elevated plasma insulin levels compared with nondiabetic patients; these patients have developed a resistance to insulin stimulation of glucose and lipid metabolism in the main insulin-sensitive tissues (muscle, liver and adipose tissues). Thus, Type 2 diabetes, at least early in the natural progression of the disease is characterized primarily by insulin resistance rather than by a decrease in insulin production, resulting in insufficient uptake, oxidation and storage of glucose in muscle, inadequate repression of lipolysis in adipose tissue, and excess glucose production and secretion by the liver. The net effect of decreased sensitivity to insulin is high levels of insulin circulating in the blood without appropriate reduction in plasma glucose (hyperglycemia). Hyperinsulinemia may be a risk factor for developing hypertension and is also thought to independently contribute to vascular disease.

Metabolic Syndrome (also termed Syndrome X) is a disorder with several phenotypes similar to those exhibited by individuals suffering from type 2 diabetes mellitus. Individuals have Metabolic Syndrome if they satisfy three or more of the following criteria: impaired fasting glucose (or glucose tolerance), low LDL levels, hypertriglyceridemia, hypertension and obesity. Additionally, patients with similar pre-diabetic states, i.e. patients with disorders similar to type 2 diabetes mellitus and Metabolic Syndrome, but not satisfying the diagnostic criteria for Metabolic Syndrome typically display diminished insulin response and aberrant glucose metabolism. Examples of individuals in such pre-diabetic states include those displaying impaired glucose tolerance with fasting blood glucose levels above average, yet less than hyperglycemic, i.e. <126 mg/dL, or displaying dyslipidemia with fasting blood triglycerides above average, yet less than hyperlipidemic, i.e. <150 mg/dL. Those with Metabolic Syndrome, as well as individuals experiencing similar pre-diabetic states, are at high risk for the development of cardiovascular disease as well as other complications of diabetes mellitus.

Because the incidence and prevalence of type 2 diabetes mellitus and Metabolic Syndrome are rapidly increasing worldwide, therapeutic control of lipid metabolism, insulin sensitivity and hypertension are critically important in the clinical management, prevention and treatment of these diseases.

Conventional treatment for type 2 diabetes mellitus has well-known limitations. Physical exercise and a reduction in dietary intake of calories can dramatically improve the diabetic condition, but compliance with this treatment is generally poor, because of well-entrenched sedentary lifestyles and excess food consumption. Clinicians are further faced with the difficult task of treating all four of the major problem areas in the diabetic: hypertension, dyslipidemia, hyperglycemia, and obesity. For treatment of hyperglycemia, the plasma level of insulin can be increased by administration of sulfonylureas (e.g. tolbutamide and glipizide), which stimulate the pancreatic β-cells to secrete more insulin, and/or by injection of insulin as the response to sulfonylureas diminishes in effectiveness and eventually fails. However, dangerously low levels of plasma glucose (hypoglycemia) can result from these last two treatments, and insulin resistance can worsen in response to increasing plasma insulin levels and weight gain. Although the biguanides can improve the response to insulin, resulting in some correction of hyperglycemia, the two biguanides, phenformin and metformin, both can produce lactic acidosis and nausea/diarrhea.

The thiazolidinediones (i.e. 5-benzylthiazolidine-2,4-diones) belong to a recently developed class of compounds, which has a novel mode of action in ameliorating many symptoms of type 2 diabetes mellitus. These agents substantially increase insulin sensitivity in muscle, liver and adipose tissue in type 2 diabetics, resulting in partial or complete correction of the elevated plasma levels of glucose typically without occurrence of hypoglycemia. Furthermore, some newly developed PPAR agonists not only improve insulin sensitivity, but also improve aspects of lipid metabolism that accompany type 2 diabetes mellitus or Metabolic Syndrome via PPAR-gamma agonism. The PPAR agonists may also alleviate disorders of lipid metabolism that accompany patients in pre-diabetic states, i.e. individuals with disorders similar to those with type 2 diabetes, but with symptoms of decreased intensity or not meeting specific criteria required to diagnose type 2 diabetes mellitus.

Disorders of lipid metabolism (dyslipidemias) include various conditions characterized by abnormal concentrations of one or more lipids (i.e. cholesterol and triglycerides), and/or apolipoproteins (i.e., apolipoproteins A, B, C and E), and/or lipoproteins (i.e., the macromolecular complexes formed by the lipid and the apolipoprotein that allow lipids to circulate in blood, such as Low Density Lipoproteins (LDL), Very Low Density Lipoproteins (VLDL) and Intermediate Density Lipoproteins (DL)). Cholesterol is primarily carried by Low Density Lipoproteins (LDL); it is commonly referred to as the “bad” cholesterol, since elevations in LDL-cholesterol correlate closely to the risk of coronary heart disease. Cholesterol is also associated with the High Density Lipoproteins (HDL), commonly referred to as the “good” cholesterol since HDL associates with cholesterol deposited in the arterial wall and atherosclerotic plaques for reverse cholesterol transport to the liver. It is therefore desirable to lower elevated levels of LDL cholesterol and to concurrently increase levels of HDL cholesterol. Generally, it has been found that increased levels of HDL are associated with a lower risk of coronary heart disease (CHD). E.g., Gordon, et al., 62 Am. J. Med. 707-714. (1977); Stampfer, et al., 325 N. England J. Med. 373-381. (1991); Kannel, et al., 90 Ann. Internal Med. 85-91. (1979). An example of an HDL raising agent is nicotinic acid, a drug with limited utility because doses effective to increase HDL are also associated with undesirable effects, such as flushing.

Dyslipidemias were originally classified according to the Fredrickson system which includes 6 phenotypes (i.e., I, IIa, IIb, III, IV and V), the most common being isolated hypercholesterolemia (or type IIa) which is usually accompanied by elevated concentrations of total and LDL cholesterol. The initial treatment for hypercholesterolemia is often diet modification, to one low in fat and cholesterol, coupled with appropriate physical exercise. However, as mentioned previously, compliance with an exercise regimen and diet therapy is poor, and thus drug therapy is often required.

A second common form of dyslipidemia is known as “mixed or combined hyperlipidemia” (i.e., IIb and III based upon the Fredrickson classification). This dyslipidemia is prevalent in individuals with type 2 diabetes mellitus, obesity and Metabolic Syndrome. In type IIb and III dyslipidemia, there are modest elevations of LDL-cholesterol, accompanied by more pronounced elevations of small dense LDL-cholesterol particles, VLDL and/or IDL (i.e., triglyceride rich lipoproteins), and total triglycerides. In addition, concentrations of HDL are often low and thus cholesterol deposits accumulate at higher rates in these individuals.

PPAR agonists form a structurally diverse group of compounds that elicit dramatic increases in the size and number of hepatic and renal peroxisomes. Peroxisome proliferation is also triggered by dietary and physiological factors such as a high-fat diet and cold acclimatization. Additionally, PPAR agonists increase the capacity of peroxisomes to metabolize fatty acids via increased expression of the enzymes of the beta-oxidation cycle. The thiazolidinediones mentioned above (5-benzylthiazolidine-2,4-diones) are generally believed to exert their effects through binding to PPAR's and controlling the transciption of genes involved in adipogenesis, insulin-sensitivity and lipid metabolism. See Hulin, et al., 2 Current Pharm. Design 85-102. (1996).

Three major sub-types of PPAR have been discovered and described: peroxisome proliferator activated receptor alpha (PPARα), peroxisome proliferator activated receptor gamma (PPARγ), and peroxisome proliferator activated receptor delta (PPARδ). PPARα is activated by a number of medium and long-chain fatty acids and fibrates, and it is involved in stimulating fatty acid oxidation and mediation of serum lipids. Indeed, fibric acid derivatives, such as clofibrate, fenofibrate, bezafibrate and gemfibrozil, each of which is a PPARα agonist, produce a substantial reduction in plasma triglycerides as well as some increase in HDL. Because the effects of the above compounds on LDL cholesterol levels are inconsistent (possibly depending upon the molecule, the model, and/or the dyslipidemic phenotype), agonists of PPARα are primarily used to treat hypertriglyceridemia (i.e., Fredrickson Type IV and V) and/or mixed hyperlipidemia (i.e., Fredrickson Type IIb and III). These fibric acid derivatives generally have low affinity for PPARγ receptors and thus do not regulate insulin sensitivity (see below).

The PPARγ receptor subtypes are involved in activating the program of adipocyte differentiation and insulin sensitivity rather than stimulating peroxisome proliferation in the liver. Prostaglandin J₂ derivatives and various long chain fatty acids have been identified as potential natural ligands of the PPARγ subtype. The 2,4-thiazolidindione (TZD) and 2,4-oxazolidinedione (OZD) based antidiabetic agents, as well as some prostanoids, have a high affinity for the PPARγ receptor. Activation of the PPARγ receptor results in increased insulin sensitivity and decreased hyperglycemia, and because of the low affinity of PPARγ activating compounds for PPARα receptors, most 2,4-TZD and 2,4-OZD-based antidiabetic agents instigate no direct effect on blood lipid profiles.

The human nuclear receptor gene PPARδ (hPPARδ) has been cloned from a human osteosarcoma cell cDNA library and is fully described by Schmidt, et al., 6 Molecular Endocrinology 1634-1641. (1992). The exact role of PPARδ is less well understood than the other PPAR sub-types.

Two TZDs (rosiglitazone and pioglitazone), currently approved for use in the treatment of diabetes, are PPARγ agonists. A third glitazone that agonizes PPARγ, troglitazone (Rezulin™), was withdrawn from the market. Recently, structurally divergent glitazones have been developed that are remarkable in their ability to activate both PPARα and γ receptors. Unlike the predecessor PPARγ selective agonists, rosiglitazone, pioglitazone and troglitazone, these newer dual agonists improve the lipid profile via the additional interaction with the PPARα receptor. Thus, a single compound, by virtue of combined PPARα/γ agonism can sufficiently treat two (hyperglycemia and dyslipidemia) of the four (hypertension, hyperglycemia and dyslipidemia) major contributors to diabetic mortality.

A promising class of glitazones with dual PPARα/γ agonist activity is described in U.S. Pat. Nos. 6,030,990, 6,001,862 and 6,147,101, assigned to Kyorin Pharmaceutical, Ltd. The compounds described in these patents are PPARα/γ dual agonists, as they activate both the PPAR α and PPAR γ sub-types. They are effective in treating elevated serum glucose and elevated lipids in patients having type 2 diabetes mellitus, and thus provide benefits beyond those of traditional TZDs, OZDs and other single PPAR agonists. Since these dual agonists can treat the specific combination of insulin-resistance coupled with poor blood-lipid profiles, they are useful to address what before required several compounds, and decrease the possibilities of drug interaction and liver toxicity.

Anti-hypertensive agents, such as those regulating the renin-angiotensin-aldosterone system (RAAS), are also of considerable therapeutic benefit in the treatment of hypertension-associated disorders such as type 2 diabetes mellitus, Metabolic Syndrome, and pre-diabetic individuals afflicted with hyperlipidemia and atherosclerosis. Indeed, Angiotensin II, the octapeptide mediator of the ubiquitous renin-angiotensin system, has strong vasoconstrictive functions and is considered to be a premier mediator of various circulatory diseases. Furthermore, atherosclerotic lesions, strong contributors to hypertension and coronary artery disease, are reported to be suppressed upon inhibition of RAAS by antagonism of Angiotensin signaling. E.g., Chobania, et al., 15 Hypertension 327-331 (1990). However, because the Angiotensin Converting Enzyme (ACE), a major target of anti-hypertensive compounds, is also responsible for suppression of the bradykinin inflammatory response, ACE antagonists are known to cause a non-productive cough. Thus, the recent development of Angiotensin II Type I receptor (A-2) inhibitors allows specific inactivation of RAAS without an inflammatory response, and provides clinicians with a method to control hypertension in diabetics, pre-diabetics, and those afflicted with Metabolic Syndrome. The first of these non-peptidic A-2 inhibitors was losartan (Cozaar®), and numerous other “sartans” such as candesartan, irbesartan, and zolasartan, have since emerged.

A generic class of A-2 antagonists is described in U.S. Pat. No. 5,138,069, assigned to E. I. Du Pont de Nemours and Company. More specific sartans are described in patents U.S. Pat. Nos. 5,266,583 and 5,264,447 assigned to Merck & Co., Inc. Because hypertension in diabetics and others suffering from the above conditions is often related to hyperlipidemia, dyslipidemia and hyperglycemia, the combination of an agent that decreases vasoconstriction (A-2 antagonists) with an agent that improves both lipid and glycemic profiles (PPAR α/γ agonists) is of immeasurable benefit. Indeed, this combination therapy and/or pharmaceutical composition addresses three (hypertension, hyperglycemia and dyslipidemia) of the contributors (hypertension, hyperglycemia and dyslipidemias) to atherosclerosis, coronary artery disease and diabetic mortality.

### SUMMARY OF THE INVENTION

A method of treating hypertension and type 2 diabetes mellitus, Metabolic Syndrome or a pre-diabetic condition, in a mammalian patient in need of such treatment, is disclosed comprising administering to said patient a dual PPARα/γ agonist and an Angiotensin II Type I receptor antagonist in an amount that is effective to treat hypertension and type 2 diabetes mellitus, Metabolic Syndrome or a pre-diabetic condition.

### DETAILED DESCRIPTION OF THE INVENTION

The present invention relates to the treatment of type 2 diabetes (non-insulin dependent diabetes mellitus, or type 2 diabetes mellitus) and to various disorders associated with type 2 diabetes mellitus, by the administration of the combination of active ingredients described below. The invention further relates to the treatment or amelioration of hypertension, hyperglycemia and hyperlipidemia associated with type 2 diabetes mellitus by administration of the combination of active ingredients described below. The invention further relates to the treatment or amelioration of hypertension, hyperglycemia, and dyslipidemia associated with a pre-diabetic condition or that are a part of Metabolic Syndrome, by administration of the combination of active ingredients described below. The invention further relates to the treatment of one or more other diseases or conditions that often accompany type 2 diabetes mellitus including lipid disorders, such as mixed or diabetic dyslipidemia, isolated hypercholesterolemia, elevated IDL-C and/or non-HDL-C, elevated hyperapo-B-liproteinemia, hypertriglyceridemia, elevated triglyceride-rich-lipoproteins, and low HDL cholesterol, by administration of the combination of active ingredients described below. The invention further relates to the treatment or amelioration of hyperglycemia, atherosclerosis and obesity by administration of the combination of active ingredients described below. The diseases listed above are treated or controlled by administration of a combination of a therapeutically effective amount of a PPARα/γ dual agonist and a therapeutically effective amount of an A-2 antagonist, including pharmaceutically acceptable salts of one or more of the active ingredients.

The combinations disclosed herein are useful in the treatment of type 2 diabetes, Metabolic Syndrome and associated disorders, and may be used as an adjunct to diet and exercise to decrease hypertension in patients who have type 2 diabetes or Metabolic Syndrome, and concurrently to control or ameliorate hyperglycemia and/or the dyslipidemia, hyperlipidemia, hypercholesterolemia and other lipid disorders that often occur in diabetic patients. In particular, the combinations are effective for treating patients with type 2 diabetes with or without Fredrickson types IIa, IIb, III, IV and V hyperlipidemia or Metabolic Syndrome. Furthermore, the combinations are useful for reducing reduce blood pressure, insulin resistance, hyperglycemia, elevated total-cholesterol, LDL-cholesterol, non-HDL-cholesterol, apolipoprotein B, and TG, and increasing serum HDL-C and Apolipoprotein A-I and A-II levels.

To summarize, the combinations as defined above are useful in treating the hypertension associated with type 2 diabetes, pre-diabetic conditions, or Metabolic Syndrome, and for concurrently treating the following conditions that often accompany the above:
(1) lipid disorders;
(2) hyperlipidemia
(3) obesity;
(4) hypercholesterolemia;
(5) hypertriglyceridemia;
(6) dyslipidemia;
(7) hyperglycemia;
(8) insulin resistance;
(9) low HDL cholesterol; and
(10) atherosclerosis and sequallae of atherosclerosis, such as angina, claudication, heart attack and stroke.

This invention encompasses a method of treating, preventing, or minimizing the risk of developing the above disorders in a patient in need of such treatment, by administering to the patient a dual PPAR α/γ agonist in combination with an A-2 antagonist, said compounds being administered in an amount that is effective to treat, prevent, or minimize the risk of developing the above disorders.

A preferred embodiment of this invention entails the use of a balanced dual PPAR α/γ agonist as described below. These dual PPAR α/γ agonists may be selected from the group consisting of: cinnamates and dihydrocinnamates, L-tyrosine derivatives, phenyl propanoic acid and other propanoic acid derivatives, propionic acid derivatives, iso-oxazolidinedione and oxazolidinedione derivatives, thiazolidinedione, tricyclics, carboxylic acids, malonic acids, oxobenzylglycine derivatives, alkanoate derivatives, benzamide derivatives, glitazones, phenyalkyloxy phenyl derivatives and isoprenols.

The most preferred dual PPAR α/γ agonist used in the invention is the benzamide derivative, KRP-297, or a pharmaceutically acceptable salt or solvent thereof.

Another preferred embodiment of the combination therapy described above entails the administration of one of the sartan class of A-2 antagonists selected from: abitesartan, benzyllosartan, elisartan, embusartan, enoltasosartan, fonsartan, forasartan, glycyllosartan, milfasartan, olmesartan, opomisartan, pratosartan, ripisartan, eprosartan, candesartan, irbesartan, saprisartan, tasosartan, telmisartan, valsartan, zolasartan and losartan. The most preferred A-2 antagonist for use in this invention is losartan, or a different salt thereof. A highly preferred embodiment of the combination therapy described in the present invention comprises administration of KRP-297, or a salt thereof, in combination with losartan.

Furthermore, one or more additional compounds may be administered along with the combination of a PPAR α/γ agonist, preferably KRP-297 or its salt, and the A-2 antagonist, preferably losartan. Examples of additional compounds that may be selected include: ACE inhibitors; insulin sensitizers including single PPARy agonists, protein tyrosine phosphatase-lB (PTP-1B) inhibitors, and dipeptidyl peptidase IV (DP-IV) inhibitors; insulin or insulin mimetics; sulfonylureas; α-glucosidase inhibitors; cholesterol lowering agents such as HMG-CoA reductase inhibitors, single PPAR α agonists, bile acid sequestrants, nicotinyl alcohol, nicotinic acid or a salt thereof, acyl CoA: cholesterol acyltransferase inhibitors, and antioxidants; PPARδ agonists; antiobesity compounds; ileal bile acid transporter inhibitors; antiinflammatory agents such as non-steroidal antiinflammatory drugs, glucocorticoids, azulfidine, cyclooxygenase 2 selective inhibitors; agents intended to inhibit platelet activation and aggregation; antihypertensives including diuretics, calcium channel blockers, β-adrenergic blockers, renin inhibitors, α-adrenergic antagonists, sympatholytic agents, atriopeptide inhibitors, serotonin inhibitors, A2-Adenosine receptor agonists, potassium channel agonists, reserpine, minoxidil, guanethidine, hydralazine hydrochloride and sodium nitroprusside.

A preferred additional agent to be administered with the present combination therapy of a dual PPAR α/γ agonist and an A-2 antagonist is an HMG CoA reductase inhibitor, preferably simvastatin.

Another preferred additional compound for use in treating, preventing, or minimizing the likelihood of developing type 2 diabetes mellitus and hypertension is ezetimibe, which blocks the absorption or reabsorption of cholesterol from the intestine. Combinations of one or more of the above agents with the combination therapy utilizing a PPAR α/γ agonist and an A-2 receptor antagonist is particularly useful for the treatment of disorders associated with type 2 diabetes mellitus, Metabolic Syndrome, and pre-diabetic states such as dyslipidemia, platelet aggregation and hyperglycemia.

Additionally, this invention includes a pharmaceutical composition that is useful for the treatment of hypertension associated with type 2 diabetes mellitus, Metabolic Syndrome, and pre-diabetic states, which is comprised of a PPAR α/γ dual agonist in combination with an A-2 agonist, and a pharmaceutically acceptable carrier. Examples of dual PPAR α/γ agonists that are useful in this regard are selected from the group consisting of: cinnamates and dihydrocinnamates, L-tyrosine derivatives, phenyl propanoic acid and other propanoic acid derivatives, propionic acid derivatives, isooxazolidinedione and oxazolidinedione derivatives, thiazolidinedione, tricyclics, carboxylic acids, malonic acids, oxobenzylglycine derivatives, alkanoate derivatives, benzamide derivatives, glitazones, phenyalkyloxy phenyl derivatives and isoprenols. Descriptions of these categories of compounds, and examples of such compounds are listed in Table 1. The classes of compounds identified in the table below are defined in accordance with the patents, primary literature and patent publications shown. Thus, for example, L-tyrosine derivatives are shown in WO 00/57001 and WO 00/18002. An example of a preferred tyrosine derivative is GW-409544.

The most preferred dual PPAR α/γ agonist used in the pharmaceutical composition is the benzamide derivative, KRP-297 or a salt thereof.

Preferred A-2 antagonists are selected from the group consisting of: abitesartan, benzyllosartan, elisartan, embusartan, enoltasosartan, fonsartan, forasartan, glycyllosartan, milfasartan, olmesartan, opomisartan, pratosartan, ripisartan, eprosartan, candesartan, irbesartan, saprisartan, tasosartan, telmisartan, valsartan, zolasartan, and losartan. A more preferred A-2 antagonist for use in this invention is losartan or a different salt thereof. A highly preferred embodiment of the pharmaceutical composition of the present invention comprises the combination of KRP-297 or a salt thereof, with losartan, or a different salt thereof.

TABLE 1
Examples of dual PPAR α/γ agonists and their origin.

| COMPOUND | DERIVATIVE | REFERENCE/ORIGIN |
|---|---|---|
| GW-409544 and | L-tyrosine | WO 00/57001, WO 00/18002 |
| salts thereof | | |
| DRF-2725; | Phenyl Propanoic Acid | US 6,054,453, WO |
| Liu, et al., Biorg | Propanoic Acid | 00/008002, WO 00/157001 |
| and Med. Chem. | | WO 02/08188 |
| Ltrs, 11, 2385 | | |
| (2001). | | |
| JTT-501 and salts | Isoxazolinediones | US 5,728,720 |
| thereof (JTT-601) | Oaxazolinediones | |
| Sauberg, et al., J. | Propionic Acids | US 6,369,067, WO |
| Med. Chem., 45, | Carboxylic Acids | 01/079150, WO 01/261654, |
| 789 (2002); | Malonic Acids | WO 01/055086, WO |
| LY-465608 | Tricyclics | 01/055085, WO 01/053257 |
| | | US 6,239,148, WO |
| | | 00/0066572, WO 00/0063209, |
| | | WO 00/0063189, WO |
| | | 00/0063153, WO 00/0050414 |
| | | WO 00/0023425, WO |
| | | 00/0023417, WO 00/0023416, |
| | | WO 00/0023415, US |
| | | 6,054,453, WO 01/016120 |
| MCC-555; | Thiazolidinediones | US 5,594,016; US 5,693,651; |
| NC-2100 | | WO 99/32465 US 6,200,998; |
| | | US 6,008,237; WO 02/26729 |
| GW-2331 | Fibrates | WO 92/10468, US 5,658,944 |
| KRP-297 | Benzamides | US 6,030,990 |
| | Glitazones | |
| AZ-242 and salts | Dihydro-cinnamates | US 6,258,850, WO |
| thereof | Cinnamates | 01/040171, WO 01/040170, |
| | Phenalkyloxy phenyl | WO 01/040169, WO |
| | | 01/040166, WO 01/040165 |
| BMS-298585 | Oxobenzylglycine | Bristol-Myers |
| | Quinoline | WO 01/070698 |
| DRF-544158 | Alkanoates | Dr. Reddy's |
| Takahashi, et al., | Isoprenols | |
| FEBS Letters, | | |
| 514, 315 (2002) | | |

Additional compounds may be admistered along with the combination of a PPAR α/γ agonist, preferably KRP-297, and an A-2 antagonist, preferably losartan. Examples of such additional compounds include: ACE inhibitors, insulin sensitizers including single PPAR y agonists, protein tyrosine phosphatase-lB (PTP-1B) inhibitors, and dipeptidyl peptidase IV (DP-IV) inhibitors; PPAR γ or α agonists; insulin or insulin mimetics; sulfonylureas; α-glucosidase inhibitors; cholesterol lowering agents such as HMG-CoA reductase inhibitors, bile acid sequestrants, nicotinyl alcohol, nicotinic acid or a salt thereof, acyl Coenzyme A: cholesterol acyltransferase inhibitors (ACAT), and antioxidants; antiobesity compounds; ileal bile acid transporter inhibitors; anti inflammatory agents such as non-steroidal antiinflammatory drugs, glucocorticoids, azulfidine and cyclooxygenase 2 selective inhibitors; agents intended to inhibit platelet activation and aggregation; antihypertensives including: diuretics, calcium channel blockers, β-adrenergic blockers, renin inhibitors, a-adrenergic antagonists, sympatholytic agents, atriopeptide inhibitors, serotonin inhibitors, A2-Adenosine receptor agonists, potassium channel agonists, reserpine, minoxidil, guanethidine, hydralazine hydrochloride and sodium nitroprusside.

A preferred additional agent to be combined with the combination of dual PPAR α/γ agonist and the A-2 antagonist is an HMG CoA reductase inhibitor, preferably simvastatin.

Another preferred additional compound for use in treating, preventing, or minimizing the likelihood of developing type 2 diabetes mellitus and hypertension is ezetimibe, which blocks the absorption or reabsorption of cholesterol from the intestine.

Combinations of one or more of the above agents with the pharmaceutical composition comprised of a PPAR α/γ agonist and an A-2 antagonist has utility for the treatment of hypertension and various disorders associated with type 2 diabetes mellitus, Metabolic Syndrome, and pre-diabetic states, such as dyslipidemia, platelet aggregation, impaired glucose tolerance and hyperglycemia.

PPAR agonists are classified as either I, K, or α/γ dual agonists, based on the relative potencies of the compounds as agonists of the PPARI and PPARK receptors. PPARγ agonists are those compounds that exhibit ≧50% of the maximal effects of rosiglitazone, a potent PPARγ agonist, on human PPARγ. PPARα agonists are those compounds that exhibit ≧50% of the maximal effects of fenofibrate, a potent

PPARα agonist, on human PPARα. Concentration potencies can be measured by using a cell-free co-activator association assay.

PPARα/γ dual agonists are compounds that exhibit both significant PPARα and PPARγ agonism wherein the half-maximal concentration potencies (EC₅₀) for activation of hPPARγ and the half-maximal concentration potencies (EC₅₀) for activation of hPPARα differ by less than 30-fold.

Compounds that exhibit significant PPARα and/or PPARγ agonism, as defined above, wherein the half-maximal concentration potencies (EC₅₀) for activation of hPPARγ and the half-maximal concentration potencies (EC₅₀) for activation of hPPARα differ by more than 30-fold are defined as selective PPARα or selective PPARγ agonists.

For example, a compound that exhibits ≧50% of the maximal effects of rosiglitazone on human PPARγ and therefore exhibits significant PPARγ agonism, and which exhibits a half-maximal concentration potency (EC₅₀) for activation of hPPARγ which is greater than 30-fold higher than its half-maximal concentration potency (EC₅₀) for activation of hPPARα is defined as a selective PPARγ agonist. Rosiglitazone is an example of such a compound. Likewise, a compound that exhibits ≧50% of the maximal effects of fenofibrate on human PPARα and therefore exhibits significant PPARα agonism, and which exhibits a half-maximal concentration potency (EC₅₀) for activation of hPPARα which is greater than 30-fold higher than its half-maximal concentration potency (EC₅₀) for activation of hPPARγ is defined as a selective hPPARα agonist. Fenofibrate is an example of such a compound.

Preferred PPARα/γ dual agonists for use in this invention are compounds that exhibit both significant PPARα and PPARγ agonism, as defined above, wherein the half-maximal concentration potencies (EC₅₀) for activation of hPPARγ and the half-maximal concentration potencies (EC₅₀) for activation of hPPARα differ by less than 20-fold.

A more preferred group of PPARα/γ dual agonists for use in this invention are compounds that exhibit both significant PPARα and PPARγ agonism, as defined above, wherein the half-maximal concentration potencies (EC₅₀) for activation of hPPARγ and the half-maximal concentration potencies (EC₅₀) for activation of hPPARα differ by less than 10-fold. These are the “balanced PPARα/γ dual agonists.”

PPARK agonists generally improve insulin sensitivity, thereby reducing the hyperglycemia that is symptomatic of type 2 diabetes. PPARI agonists improve lipid metabolism by lowering triglycerides, lowering LDL, and potentially raising HDL. PPARI/K dual agonists can thus control or ameliorate the hyperglycemia and dyslipidemia associated with type 2 diabetes. The preferred PPARI/K dual agonists for combination therapy with antihypertensive agents in this invention are “balanced” PPARI/K dual agonists, as defined above. These have approximately equal potencies (within a factor of 10) for agonism of both the PPARI and PPARK receptor sub-types.

Examples of PPARα/γ dual agonists are disclosed and claimed in the Kyorin patents cited above, such as U.S. Pat. No. 6,030,990. Other known compounds having dual PPARα/γ agonism activity are exemplified in Table 1, and include: dihydrocinnamate derivatives such as (S)-2-ethoxy-3-[4-[2-(4-methylsulfonyloxyphenyl)ethoxy]phenyl]-propanoicacid (AZ-242); certain fibrates such as 2-(4-[2-(3-[2,4-Difluorophenyl]-1-heptylureido) ethyl]phenoxy)-2-methylbutyric acid (GW-2331); isoprenols such as farnesol as described by Takahashi, et al,. FEBS Letters 514, 315 (2002); L-tyrosine derivatives such as N-[(1Z)-1-methyl-3-oxo-3-phenyl-lpropenyl]-O-[2-(5-methyl-2-phenyl-4-ozazolyl)ethyl]-L-Tyrosine (GW-409544); phenyl propanoic acid derivatives such as (±)3-[4-[2-(Phenoxazin-10-yl)ethoxy]phenyl]-2-ethoxypropanoic acid (DRF-2725) and those described in Liu, et al., Biorg. And Med. Chem. Letters, 11, 2385 (2001);

propionic acid derivatives such as LY4-65608 and those described by Sauberg, et al., J. Med. Chem. 45,789-804, (2002); certain isoxazolinedione derivatives such as 4-[4-[2-(5-methyl-2-phenyl-4-oxazolyl)ethoxy]benzyl]-3,5-isoxazolidinedione (JTT-501) and their salts such as JTT-601; dual PPARα/γ glitazones such as (+)-5-[[6-(2-fluorbenzyl)-oxy-2-naphy]methyl]-2,4-thiazolidinedione (MCC-555) and (±)-5-[(7-benzyloxo-3-quinolyl)methyl]-2,4-thiazolidinedione (NC-2100) described by Fulkul, et al., Diabetes, 49, 759 (2000); alkanoate derivatives such as DRF-544158; oxobenzyl glycine derivatives such as BMS-298585; and additionally carboxylic acids, malonic acids and tricyclics listed in Table 1.

Preferred compounds for use in the present invention are dual PPARα/γ agonists that exhibit both significant PPARα and PPARγ agonism, as defined above, wherein the half-maximal concentration potencies (EC₅₀) for activation of hPPARγ and the half-maximal concentration potencies (EC₅₀) for activation of hPPARα differ by less than 30-fold.

More preferred compounds for use in the present invention are dual PPARα/γ agonists that exhibit both significant PPARα and PPARγ agonism, as defined above, wherein the half-maximal concentration potencies (EC₅₀) for activation of hPPARγ and the half-maximal concentration potencies (EC₅₀) for activation of hPPARα differ by less than 20-fold.

Even more preferred compounds for use in the present invention are balanced dual PPARα/γ agonists which are compounds that exhibit both significant PPARα and PPARγ agonism, as defined above, wherein the half-maximal concentration potencies (EC₅₀) for activation of hPPARγ and the half-maximal concentration potencies (EC₅₀) for activation of hPPARα differ by less than 10-fold and include the N-benzyldioxothiazolidylbenzamide derivatives disclosed in U.S. Pat. Nos. 6,001,862 and 6,030,990.

The most preferred balanced PPARα/γ dual agonist for this invention is disclosed in Example 39 of U.S. Pat. No. 6,030,990. This compound is known as KRP-297, or 5-[(2,4-dioxo-5-thiazolidinyl)methyl]-2-methoxy-N-[[4-(trifluoromethyl)phenyl]methyl]-benzamide. The structure is shown below:

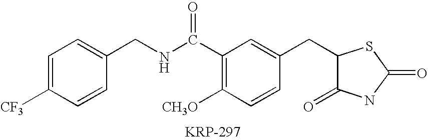

The PPARα/γ dual agonists of this invention are used in combination with an anti-hypertensive selected from the group consisting of A-2 antagonists. A-2 antagonists interfere with the signaling of the renin-angiotensin system, resulting in vasodilation and thus decreased blood pressure. The combination of A-2 antagonists and PPARI/K dual agonists has the effect of reducing blood pressure and improving the poor lipid profiles and hyperglycemia found in pre-diabetics, diabetics and those afflicted with Metabolic Syndrome. This combination is also of therapeutic benefit in the treatment of artherosclerosis and other diseases accompanied by dyslipidemia and hypertension.

Examples of A-2 antagonists are disclosed and claimed in U.S. Pat. No. 5,138,069. Preferred A-2 antagonists for use in the present invention are “sartans” such as abitesartan, benzyllosartan, elisartan, embusartan, enoltasosartan, fonsartan, forasartan, glycyllosartan, milfasartan, olmesartan, opomisartan, pratosartan, ripisartan, eprosartan, candesartan, irbesartan, saprisartan, tasosartan, telmisartan, valsartan, zolasartan, etc.

More preferred A-2 antagonists for use in the present invention are the non-peptide substituted imidazole derivatives disclosed in U.S. Pat. No 5,138,069.

The most preferred A-2 antagonist for this invention is the substituted imidazole disclosed in U.S. Pat. No 5,266,583. This compound is known generally as losartan (Cozaar®), or 2-butyl-4-chloro-1-[p-(o-1H-tetrazol-5-ylphenyl)-benzyl]imidazole-5-methanol provided in the form of the monopotassium salt. The structure is shown below:

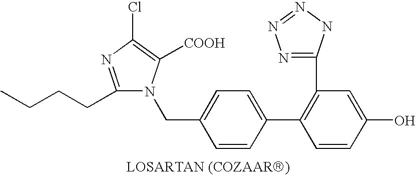

The use of the above combinations is beneficial to a patient with hypertension and hyperglycemia and/or insulin resistance and/or lipid disorders such as hypertriglyceridemia, hypercholesterolemia, hyperlipidemia, atherosclerosis and dyslipidema.

“Combination” therapy or a drug “combination” means that two or more active components are administered to a patient at approximately the same time or at times that are sufficiently close that both drugs will be present in the patient at a level sufficient to be therapeutic or sub-therapeutic at certain times of the day. Combination therapy can also occur when the two medicines are administered at the same time or at different times during the day. Combination therapy thus also includes therapies in which the two active drugs are administered on different overlapping schedules. A pharmaceutical composition containing the two drugs is preferred when practicable.

The term “pharmaceutical composition”, encompasses the active ingredient(s) and the carrier, as well as any product which results, directly or indirectly, from the combination, complexation or aggregation of any of the ingredients. Accordingly, the pharmaceutical compositions of the present invention encompass composition made by admixing the compounds of the present combination and the pharmaceutically acceptable carrier.

Optical Isomers—Diastereomers—Geometric Isomers—Tautomers

The active ingredients in the combinations of this invention may contain one or more asymmetric centers. The individual active components can thus occur as racemic mixtures, single enantiomers, diastereomeric mixtures and individual diastereomers. The present invention includes all combinations of such isomeric forms.

Some of the compounds described herein may exist as tautomers, such as ketones and enols. The individual tautomers as well as mixtures thereof are encompassed with the compounds that are described herein.

Salts

The term “pharmaceutically acceptable salts” refers to salts prepared from pharmaceutically acceptable non-toxic bases or acids including inorganic or organic bases and inorganic or organic acids. Since losartan is available commercially as a potassium salt, it is possible, and within the scope of the present invention, to provide and utilize an alternative salt-based form. Salts derived from inorganic bases include aluminum, ammonium, calcium, copper, ferric, ferrous, lithium, magnesium, manganic salts, manganous, potassium, sodium, zinc and the like. Particularly preferred are the ammonium, calcium, magnesium, potassium and sodium salts. Salts in the solid form may exist in more than one crystal structure, and may also be in the form of hydrates. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When the compounds used in the present invention are basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particularly preferred are citric, hydrobromic, hydrochloric, maleic, phosphoric, sulfuric and tartaric acids.

It will be understood that, as used herein, references to the compounds used in the combinations described herein are meant to also include the pharmaceutically acceptable salts.

Administration and Dose Ranges

Any suitable route of administration may be employed for providing a human patient with an effective dose of the compounds used in the present invention. For example, oral, rectal, topical, parenteral, ocular, pulmonary, nasal and the like may be employed. Dosage forms include tablets, troches, dispersions, suspensions, solutions, capsules, creams, ointments, aerosols and the like. Preferably the method of administration is oral.

The effective dosages employed may vary depending on the particular compounds employed, the mode of administration, the condition being treated and the severity of the condition being treated. Such dosages may be readily ascertained by a person skilled in the art.

When treating diabetes mellitus and/or hyperglycemia or hypertriglyceridemia or other diseases for which the compounds described herein are indicated, generally satisfactory results are obtained when the compounds of the present invention are administered at normal daily doses, from about 0.1 milligram to about 100 milligram per kilogram of body weight, preferably given as a single daily dose or in divided doses two to six times a day, or in sustained release form. For most patients, the total daily dosage for these drugs ranges from about 0.1 milligrams to about 1000 milligrams, preferably from about 1 milligram to about 50 milligrams. In the case of a 70 kg adult human, the total daily dose of each compound will generally be from about 1 milligrams to about 350 milligrams. This dosage regimen may be adjusted by the treating physician to provide the optimal therapeutic response for the patient, taking into account the specific compounds that are used.

Preferred examples of daily dosages of KRP-297 and losartan include, for example: for KRP-297, 1 mg, 3 mg, 5 mg, 10 mg or 20 mg; and for losartan (Cozaar®), 1 mg, 5 mg, 10 mg, 20 mg, 50 mg or 100 mg.

Pharmaceutical Compositions

The pharmaceutical compositions described herin comprise the compounds described above and a pharmaceutically acceptable carrier. The compositions are typically suitable for oral, rectal, topical, parenteral (including subcutaneous, intramuscular, and intravenous), ocular (ophthalmic), or pulmonary (nasal or buccal inhalation) administration. The most suitable route in any given case will depend on the nature and severity of the condition being treated and on the nature of the active ingredients. They may be conveniently presented in unit dosage form and compounded by methods well-known in the art of pharmacy.

The compounds of this invention can be combined in intimate admixture with a pharmaceutical carrier according to conventional pharmaceutical compounding techniques, or they can be combined together in a carrier. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets, with the solid oral preparations being preferred over the liquid preparations.

Because of their ease of administration, tablets and capsules represent the most advantageous oral dosage forms. If desired, tablets may be coated by standard aqueous or nonaqueous techniques. The percentage of active compound in these compositions may, of course, be varied and may conveniently be between about 1 percent to about 90 percent of the unit on a w/w basis.

The tablets, capsules and the like also typically include a binder such as gum tragacanth, acacia, corn starch or gelatin; excipients such as dicalcium phosphate; a disintegrating agent such as corn starch, potato starch, alginic acid; a lubricant such as magnesium stearate; and a sweetening agent such as sucrose, lactose or saccharin in the carrier material.

Various coatings may be included. For example, tablets may be coated with shellac, sugar or both. Syrups or elixirs may contain, sucrose as a sweetening agent, methyl and propylparabens as preservatives, dyes and flavorings such as cherry or orange flavor.

Parenteral solutions or suspensions of these active compounds can be prepared in water suitably mixed with a surfactant such as hydroxypropylcellulose. Dispersions can also be prepared in glycerol, liquid polyethylene glycols and mixtures thereof in oils. Under ordinary conditions of storage and use, these preparations maycontain preservatives to prevent the growth of microorganisms.

Injectables also include sterile aqueous solutions or dispersions and sterile powders for the extemporaneous preparation of sterile injectable solutions or dispersions. The carrier can be a solvent or dispersion medium containing, for example, water, ethanol, polyol (e.g. glycerol, propylene glycol and liquid polyethylene glycol), suitable mixtures thereof and vegetable oils.

Combinations with a Third Therapeutic Compound

The invention optionally includes other drugs that may be useful in the treatment, suppression or amelioration of the diseases or conditions for which the combinations of this invention are useful. Such other drugs may be administered contemporaneously or sequentially with the combination described above. It is also contemplated that when used in combination with one or more other active ingredients, the compounds of the present invention and the other active ingredients may be used in lower doses than when each is used alone. Accordingly, the pharmaceutical compositions of the present invention include those that contain one or more other active ingredients, in addition to the compounds of the present combination.

Examples of other active ingredients that may be administered with the combination of drugs of this invention, separately or in the same pharmaceutical composition, include:

(a) insulin sensitizers including (i) single PPARy agonists such as the glitazones (e.g. troglitazone, pioglitazone, englitazone, rosiglitazone, and the like), and compounds disclosed in WO97/27857, 97/28115, 97/28137 and 97/27847; (ii) biguanides such as metformin and phenformin; (iii) protein tyrosine phosphatase-1B (PTP-1B) inhibitors, and (iv) dipeptidyl peptidase IV (DP-IV) inhibitors;

(b) insulin or insulin mimetics;

(c) sulfonylureas such as tolbutamide and glipizide;

(d) α-glucosidase inhibitors (such as acarbose);

(e) cholesterol lowering agents such as (i) HMG-CoA reductase inhibitors (lovastatin, simvastatin, pravastatin, fluvastatin, atorvastatin, rivastatin, itavastatin, ZD4522 and other statins), (ii) sequestrants (cholestyramine, colestipol, and dialkylaminoalkyl derivatives of a cross-linked dextran), (iii) nicotinyl alcohol, nicotinic acid or a salt thereof, (iv) acyl CoA:cholesterol acyltransferase inhibitors, such as for example avasimibe, (v) PPAR α agonists, and (vi) anti-oxidants, such as probucol;

(f) PPARδ agonists such as those disclosed in WO 97/28149;

(g) antiobesity compounds (anorectics) such as fenfluramine, dexfenfluramine, phentermine, sibutramine, mazindol, orlistat, lipase inhibitors, neuropeptide Y5 inhibitors, melanocortin-4-receptor agonist, thyroid receptor beta drug, anorectic agent, CCKA agonist, leptin inhibitors and β₃ adrenergic receptor agonists;

(h) an ileal bile acid transporter inhibitor; and

(i) agents intended for use in inflammatory conditions such as aspirin, non-steroidal anti-inflammatory drugs, glucocorticoids, azulfidine and cyclo-oxygenase 2 selective inhibitors; and

(j) agents intended to inhibit platelet activation and aggregation such as cliopidogrel and ticlopidine; and

(k) ACE inhibitors;

(l) diuretics, such as amiloride, chlorothiazide, benzthiazide, ticrynafen, acetazolamide, cyclothiazide, trichloromethiazide, cyclopentiazide, hydrochlorothiazide, methyclothiazide, hydrochlorothiazide derivatives, penfluthiazide, ethiazide, hydroflumethiazide, polythiazide, chlophenamide, chlorthalidone, cyclothiazide, flumethiazide derivatives, meticrane, tripamide, metrazone, indapamide, quinethazone, furosernide, bumetanide, mefruside, azosemide, ethacrynic acid, sodium ethacrynate, piretanide, potassium canrenoate, quinethazone, triamterene, methylclothiazide, atriopeptin and spironolactone.

(m) calcium channel blockers, such as diltiazem hydrochloride, teloridine hydrochloride, nicardipine hydrochloride, varnidipine hydrochloride, flunarizine hydrochloride, verapamil hydrochloride, manidipine hydrochloride, cinnarizine, nisoldipine, nitrendipine, nifedipine, nilvadipine, felodipine, nildipine, nimodipine, penidipine, benidipine, amlodipine, isradipine and verapamil; and

(n) β-adrenergic blockers, such as timolol, atenolol, metoprolol, propanolol, nadolol and pindolol;

(o) anti-hypertensive compounds selected from:
(i) renin inhibitors such as A-69729, FK906 and FK 744; and
(ii) α adrenergic antagonists such as prazosin, doxazosin, and terazosin; and
(iii) sympatholytic agents such as methlydopa, clonidine and guanabenz; and
(iv) atriopeptide inhibitors (alone or with ANP) such as UK-79300; and
(v) serotonin antagonists such as ketanserin; and
(vi) A2-Adenosine receptor agonists such as CGS 22492C; and
(vii) potassium channel agonists such a pinacidil and cromakalim; and
(viii) reserpine, minoxidil, guanethidine, hydralazine hydrochloride and sodium nitroprusside.

A dual PPAR α and γ agonist, particularly a balanced dual PPAR agonist, has not been disclosed in combination with an A-2 antagonist for the treatment of hypertension and type 2 diabetes mellitus, Metabolic Syndrome or a pre-diabetic condition. Prior combinations provide only partial therapy for type 2 diabetes mellitus, and unlike the present invention, require inclusion of additional compoundsm to regulate lipid metabolism.

The combination of A-2 antagonists and PPAR agonists is expected to produce a synergy not exhibited by other drug combinations because recent studies indicate that dual PPAR agonism results in decreased transcription of the type 1 Angiotensin II receptor. Sugawara, et al., Endocrinology. 142, 3125-34 (2001). Thus, a dual PPAR agonist/A-2 antagonist combination, in addition to regulating blood-insulin and blood-lipid levels, can reduce hypertension via modification of the type I Angiotensin II receptor at both transcriptional and functional levels.

Finally, many current methods do not treat the specific afflicition of hypertension and type 2 diabetes mellitus, Metabolic Syndrome or pre-diabetic state, i.e. hypertension accompanying dyslipidemia and/or insulin-resistance, but rather address disorders such as artherosclerosis, nephropathy, cerebrovascular and peripheral circulatory dysfunction, obesity, hyperglycemia or ketoacidosis. Thus, these inventions do not identify the pressing need to treat hypertension associated with type 2 diabetes mellitus, an extremely common comorbidity, affecting 20-60 % of diabetics and contributing to greater than 80 % of diabetic deaths. Arauz-Pacheco, et al., 25 Diabetes Care 134-147 (2002). The current invention identifies a needed, and highly efficient method to treat the specific and common occurrence of hypertension with type 2 diabetes mellitus, Metabolic Syndrome or pre-diabetes, by combining the insulin and lipid improving effects of a balanced dual PPAR α/γ agonist with the anti-hypertensive effect of an A-2 antagonist.

In Vivo Assays

Male db/db mice (10-11 week old C57B1/KFJ, Jackson Labs, Bar Harbor, Me.) were housed 5/cage and allowed ad lib. access to ground Purina rodent chow and water. The animals, and their food, were weighed every 2 days and were dosed daily by gavage with vehicle (0.5% carboxymethylcellulose)±test compound at the indicated dose. Drug suspensions were prepared daily. Plasma glucose, and triglyceride concentrations were determined from blood obtained by tail bleeds at 3-5 day intervals during the study period. Glucose and triglyceride determinations were performed on a Boehringer Mannheim Hitachi 911 automatic analyzer (Boehringer Mannheim, Indianapolis, IN) using heparinized plasma diluted 1:6 (v/v) with normal saline. Lean animals were age-matched heterozygous mice maintained in the same manner.

Male Golden Syrian hamsters weighing ˜150 g are used to measure lipid modulation effects of test compounds. Hamsters are housed in boxes (5 per box), are fed a normal rodent chow diet, and are given free access to water. Compounds are suspended in 0.5% methylcellulose and gavaged daily to the hamsters for 9 days (10 hamsters per group). On the morning of the 10^{th} day, the hamsters are euthanized with carbon dioxide and blood samples are obtained via heart puncture. Serum levels of total cholesterol and triglycerides are determined.

Mature male beagle dogs, weighing ˜15 kg on average, are used to measure the lipid modulation effects of test compounds. Dogs are housed individually, are fed a cholesterol-free chow diet, and are given free access to water. Prior to the start of experiments, samples are taken weekly from the jugular vein and the serum cholesterol levels are determined. To test the effects of compounds on serum cholesterol, compounds are suspended in 0.5% methylcellulose and gavaged daily to the dogs for 2 weeks (5 dogs per group). Blood samples are taken during and after the dosing period, and serum levels of total cholesterol and triglycerides are determined.

Cell-Free Co-Activator Association Assay

This assay measures the ability of compounds to promote the association of PPARy (or its isolated ligand binding domain) or PPARα (or its isolated ligand binding domain) with a protein (or portion of a protein) that is (or is derived from) a co-activator molecule such as Creb Binding Protein (CBP) or Steroid Receptor Coactivator 1 (SRC-1) and can be used to identify compounds with both PPARα and PPARγ agonist activity. This assay is described in Zhou et al. Nuclear receptors have distinct affinities for co-activators: characterization by fluorescence resonance energy transfer. Mol Endocrinol, 12, 1594-1604 (1998).

Human PPARα and PPARγ Binding Assays

An alternative to measuring agonist activity of compounds in cell-based transactivation assays or cell-free co-activator association assays is to determine that compounds can function as ligands by binding to both PPARγ and PPARα. Compounds with half-maximal concentration potencies (IC₅₀'s or KI's) for displacement of radioligand binding to hPPARγ vs. hPPARα that differ by less than 30-fold and preferably less than 10-fold can be considered as dual ligands. For these assays, the methods described below can be employed (as also described in: Berger, et. al. Novel peroxisome proliferator-activated receptors (PPARγ) and PPARδ ligands produce distinct biological effects, J Biol Chem, 274, 6718-6725 (1999)).

Human PPARK2 and human PPARI were expressed as a GST-fusion protein in E. coli. The full-length human cDNA for PPARK₂ was subcloned into the pGEX-2T expression vector (Pharmacia). The full-length human cDNA for PPARI was subcloned into the pGEX-KT expression vector (Pharmacia). E. coli containing the respective plasmids were propagated, induced, and harvested by centrifugation. The resuspended pellet was broken in a French press and debris was removed by centrifugation at 12,000×g. Recombinant human PPAR receptors were purified by affinity chromatography on glutathione sepharose. After application to the column, and one wash, receptor was eluted with glutathione. Glycerol (10%) was added to stabilize the receptor and aliquots were stored at −80° C.

For each assay, an aliquot of receptor was incubated in TEGM (10 mM Tris, pH 7.2, 1 mM EDTA, 10% glycerol, 7 μL/100 ml β-mercaptoethanol, 10 mM Na molybdate, 1 mM dithiothreitol, 5 μg/mL aprotinin, 2 μg/mL leupeptin, 2 μg/mL benzamidine and 0.5 mM PMSF) containing 0.1% non-fat dry milk and 10 nM [³H₂] L-746,962 (21 Ci/mmole) which binds the PPARγ₂ receptor, +test compound. Assays were incubated for ˜16 hr at 4° C. in a final volume of 150 μL. Unbound ligand was removed by incubation with 100 μL dextran/gelatin-coated charcoal, on ice, for 10 min. After centrifugation at 3000 rpm for 10 min at 4° C., 50 μL of the supernatant fraction was counted in a Topcount. In this assay the KD for L-746,962 is ≅1 nM.

For a human PPARα binding assay, an aliquot of receptor was incubated in TEGM (10 mM Tris, pH 7.2, 1 mM EDTA, 10% glycerol, 7 μL/100 ml β-mercaptoethanol, 10 mM Na molybdate, 1 mM dithiothreitol, 5 μg/mL aprotinin, 2 μg/mL leupeptin, 2 μg/mL benzamide and 0.5 mM PMSF) containing 0.1% non-fat dry milk and 5.0 nM [³H₂]L-783483 which binds the PPARα receptor, ±test compound. Assays were incubated for ˜16 hr at 4° C. in a final volume of 150 μL. Unbound ligand was removed by incubation with 100 μL dextran/gelatin-coated charcoal, on ice, for ˜10 min. After centrifugation at 3000 rpm for 10 min at 4° C., 50 μL of the supernatant fraction was counted in a Topcount.

Cell Proliferation Assay

This assay measures the ability of cells to convert MTS tetrazolium into formazan, using the AQᵤₑₒᵤₛ cell proliferation assay kit (Promega, Madison, Wis.). This conversion is presumably accomplished by NADPH or NADH produced by dehydrogenase enzymes in metabolically active cells. The assay is described in Shu, et al., Biochem Biophys Res Comm, 267, 345-349 (2000).

### EXAMPLES

The following examples are provided to illustrate the invention and are not to be construed as limiting the invention in any manner. The scope of the invention is defined in the appended claims.

1. Capsules

| | | |
|---|---|---|
| (1) KRP-297 | 3-5 | mg |
| (2) losartan (Cozaar ® ) | 50 | mg |
| (3) lactose | 19 | mg |
| (4) microcrystalline cellulose | 70 | mg |
| (5) magnesium sterate | 10 | mg |
| one capsule | 150 | mg |

(1), (2), (3), (4), and ½ of (5) were mixed and then granulated. To the granules was added the remainder of (5), and the while was filled into a gelatin capsule.

2. Tablets

| | | |
|---|---|---|
| (1) KRP-297 | 3-5 | mg |
| (2) losartan (Cozaar ® ) | 50 | mg |
| (3) Lactose | 46.4 | mg |
| (4) corn starch | 20 | mg |
| (5) polyethylene glycol | 2.6 | mg |
| (6) hydroxypropyl cellulose | 4 | mg |
| (7) carmellose calcium | 5.6 | mg |
| (8) magnesium sterate | 0.4 | mg |
| one tablet | 130 | mg |

(1), (2), (3), (4), (5), ⅔ of (6), ⅔ of (7) and ½ of (8) were mixed and then granulated. To the granules were added the remainders of (6), (7), and (8), followed by subjecting the mixture to compression molding.

3. Injection

| | | | |
|---|---|---|---|
| (1) | KRP-297 | 3-5 | mg |
| (2) | losartan (Cozaar ®) | 50 | mg |
| (3) | inositol | 59 | mg |
| (4) | benzyl alcohol | 20 | mg |
| | one ampoule | 130 | mg |

(1), (2), (3), and (4) were dissolved in distilled water for injection to make the whole volume 2 ml, which was filled into an ampoule. The whole process was conducted under sterile conditions.

While certain preferred embodiments have been described herein in detail, numerous alternative embodiments are contemplated as falling within the scope of the invention. All references provided herein are hereby incorporated by reference.

1. A method of treating hypertension and type 2 diabetes mellitus, Metabolic Syndrome or a pre-diabetic condition, in a mammalian patient in need of such treatment, comprising administering to said patient a dual peroxisome proliferator activated receptor alpha/gamma (PPARα/γ) agonist and an Angiotensin II Type I receptor (A-2) antagonist in an amount effective to treat hypertension and type 2 diabetes mellitus, Metabolic Syndrome or a pre-diabetic condition.

2. A method as recited in claim 1, wherein the dual PPAR α/γ agonist is a balanced dual PPAR α/γ agonist.

3. A method as recited in claim 2, wherein the dual PPAR α/γ agonist is selected from the group consisting of: dihydrocinnamate and cinnamate derivatives, L-tyrosine derivatives, phenyl propanoic acid and propanoic acid derivatives, isoxazolidinedione and oxazolidinedione derivatives, thiazolidinediones, tricyclics, carboxylic acid and malonic acid derivatives, oxobenzylglycine derivatives, fibrates, quinoline derivatives, alkanoate derivatives, phenyalkyloxy phenyl derivatives, benzamide derivatives and isoprenols, including pharmaceutically acceptable salts of one or more of the active ingredients.

4. A method as recited in claim 3, wherein the dual PPAR α/γ agonist is selected from the group consisting of GW-409544, DRF-2725, JTT-501, LY-465608, MCC-555, NC-2100, GW-2331, AZ-242, BMS-298585, DRF-544158, and KRP-297, or a pharmaceutically acceptable salt thereof.

5. A method as recited in claim 4, wherein A-2 antagonist is selected from the group consisting of: losartan, abitesartan, benzyllosartan, elisartan, embusartan, enoltasosartan, fonsartan, forasartan, glycyllosartan, milfasartan, olmesartan, opomisartan, pratosartan, ripisartan, eprosartan, candesartan, irbesartan, saprisartan, tasosartan, telmisartan, valsartan, and zolasartan or the pharmaceutically acceptable salts or solvates thereof.

6. A method as recited in claim 5, wherein the A-2 antagonist is losartan or a pharmaceutically acceptable salt-based alternative thereof.

7. A method as recited in claim 5 further comprising administering to the patient a compound selected from the group consisting of: ACE inhibitors, insulin sensitizers including single PPARyagonists, protein tyrosine phosphatase-IB inhibitors, dipeptidyl peptidase IV inhibitors, insulin or insulin mimetics; sulfonylureas; α-glucosidase inhibitors; cholesterol lowering agents selected from single PPAR α agonists, bile acid sequestrants, nicotinyl alcohol, nicotinic acid or a salt thereof, acyl Coenzyme A:cholesterol acyltransferase inhibitors, and antioxidants; PPARδ agonists; antiobesity compounds; ileal bile acid transporter inhibitors; antiinflammatory agents selected from aspirin, non-steroidal anti-inflammatory drugs, glucocorticoids, azulfidine and cyclooxygenase-2 selective inhibitors; agents intended to inhibit platelet activation and aggregation; antihypertensives selected from:calcium channel blockers, β-adrenergic blockers, renin inhibitors, α-adrenergic antagonists, sympatholytic agents, atriopeptide inhibitors, serotonin inhibitors, A2-Adenosine receptor agonists, potassium channel agonists, reserpine, minoxidil, guanethidine, hydralazine hydrochloride and sodium nitroprusside.

8. A method in accord with claim 7 wherein the HMG-CoA reductase inhibitor is simvastatin, or a pharmaceutically acceptable salt or solvate thereof.

9. A method in accord with claim 7 wherin the compound administered is ezetimibe, or a pharmaceutically acceptable salt or solvate thereof.

10. A method of treating, delaying or ameliorating hypertension and type 2 diabetes mellitus, Metabolic Syndrome or a pre-diabetic state and further treating, delaying or ameliorating a lipid disorder selected from dyslipidemia, hyperlipidemia and hypercholesterolemia in a patient in need of such treatment, comprising administering losartan and a compound selected from the group consisting of GW-409544, DRF-2725, JTT-501, LY-465608, MCC-555, NC-2100. GW-2331, AZ-242. BMS-298585, DRF-544158, and KRP-297 or a pharmaceutically acceptable salt thereof and losartan in an amount that is effective to treat, delay or ameliorate hypertension and type 2 diabetes mellitus, Metabolic syndrome or a pre-diabetic state and said lipid disorder.

11. A pharmaceutical composition comprising a dual PPAR α/γ agonist and an Angiotensin II type I receptor antagonist in combination with a pharmaceutically acceptable carrier.

12. A pharmaceutical composition in accordance with claim 11, wherein the dual PPAR α/γ agonist is a balanced dual PPAR α/γ agonist.

13. A pharmaceutical composition in accordance with claim 12, wherein the Angiotensin II Type I receptor antagonist is selected from the group consisting of: losartan, abitesartan, benzyllosartan, elisartan, embusartan, enoltasosartan, fonsartan, forasartan, glycyllosartan, milfasartan, olmesartan, opomisartan, pratosartan, ripisartan, eprosartan, candesartan, irbesartan, saprisartan, tasosartan, telmisartan, valsartan and zolasartan, and the pharmaceutically acceptable salts or solvates thereof.

14. A pharmaceutical composition as recited in claim 13, wherein the Angiotensin II Type I receptor antagonist is losartan.

15. A pharmaceutical composition in accordance with claim 12, wherein the dual PPAR α/γ agonist is selected from the group consisting of: dihydro-cinnamate and cinnamate derivatives, L-tyrosine derivatives, phenyl propanoic acid and propanoic acid derivatives, isooxazolidinedione and oxazolidinedione derivatives, thiazolidinediones, tricyclic derivatives, carboxylic acid and malonic acid derivatives, oxobenzylglycine derivatives, fibrates, quinoline derivatives, alkanoate derivatives, phenyalkyloxy phenyl derivatives, benzamide derivatives and isoprenols, including pharmaceutically acceptable or solvates thereof.

16. A pharmaceutical composition as recited in claim 15, wherein the dual PPAR α/γ agonist is selected from the group consisting of GW-409544, DRF-2725, JTT-501, LY-465608, MCC-555, NC-2100, GW-2331, AZ-242, BMS-298585, DRF-544158, and KRP-297 or a pharmaceutically acceptable salt or solvate thereof.

17. A pharmaceutical composition as recited in claim 12 further comprised of a member selected from the group consisting of: ACE inhibitors, insulin sensitizers selected from single PPARyagonists, protein tyrosine phosphatase-IB inhibitors, and dipeptidyl peptidase IV inhibitors; insulin or insulin mimetics; sulfonylureas; α-glucosidase inhibitors; cholesterol lowering agents selected from HMG-CoA reductase inhibitors, single PPAR α agonists, bile acid sequestrants, nicotinyl alcohol, nicotinic acid or a salt thereof, acyl Coenzyme A:cholesterol acyltransferase inhibitors, and anti-oxidants; PPARδ agonists; antiobesity compounds; ileal bile acid transporter inhibitors; anti-inflammatory agents such as aspirin, non-steroidal anti-inflammatory drugs, glucocorticoids, azulfidine, and cyclo-oxygenase 2 selective inhibitors; agents intended to inhibit platelet activation and aggregation; additional anti-hypertensives including: single PPAR α agonists, calcium channel blockers, β-adrenergic blockers, renin inhibitors, α-adrenergic antagonists, sympatholytic agents, atriopeptide inhibitors, serotonin inhibitors, A2-Adenosine receptor agonists, potassium channel agonists, reserpine, minoxidil, guanethidine, hydralazine hydrochloride and sodium nitroprusside, including pharmaceutically acceptable salts or solvates thereof.

18. A pharmaceutical composition as recited in claim 17 where the HMG CoA Reductase inhibitor is simvastatin, including pharmaceutically acceptable salt or solvates thereof.

19. A pharmaceutical composition in accordance with claim 12, further comprising ezetimibe or a pharmaceutically acceptable salt or solvate thereof.
